(19) United States
(12) Patent Application Publication
(10) Pub. No.: US 2018/0071493 A1
(43) Pub. Date: Mar. 15, 2018

(54) SUTURE-BASED CATHETER SECUREMENT DEVICE AND METHOD

(71) Applicant: JOSEPH MICHAEL KONSTANTARAKIS, Laguna Nigel, CA (US)

(72) Inventor: JOSEPH MICHAEL KONSTANTARAKIS, Laguna Nigel, CA (US)

(21) Appl. No.: 15/553,136

(22) PCT Filed: Feb. 23, 2016
(86) PCT No.: PCT/US16/19191

§ 371 Date: Aug. 23, 2017

Related U.S. Application Data
(60) Provisional application No. 62/119,381, filed on Feb. 23, 2015.

(51) Int. Cl. A61M 25/02; A61M 25/00

(52) U.S. Cl. CPC A61M 25/02; A61B 17/0467; A61M 2025/0286; A61M 25/0097

(57) ABSTRACT

An improved device for securing a catheter to a human comprising multiple sutures for quick securement via a novel needle arrangement.

### CROSS-REFERENCE TO RELATED APPLICATION

This is a national phase application of International Application No. PCT/US16/19191, filed Feb. 23, 2016, which claims priority to U.S. Provisional Application No. 62/119,381 dated Feb. 23, 2015 the contents of which are hereby incorporated by reference.

### FIELD OF THE INVENTION

The present invention relates to novel devices and methods for securing catheters to the skin using needles and suture.

### BACKGROUND OF THE INVENTION

Among the last steps after catheter insertion through the skin involves securing the catheter in order to prevent its migration or accidental removal. Various ways of securing a catheter to the skin have been employed. Among the most common involves devices which attach to the catheter and adhere to the skin by means of an adhesive, thus anchoring the catheter to the skin noninvasively. The advantages of this approach are that it is rapid and does not involve needle penetration of the skin. However, there are several notable disadvantages.

One disadvantage is the anchoring can be suboptimal, increasing the likelihood of catheter movement or accidental removal, particularly in instances where the adhesive is compromised such as on wet, oily, or perspiring skin. This can be particularly detrimental to a patient. For instance, if a pleural chest drain is placed and sub-optimally secured, it can be accidentally pulled out, placing the patient at risk for a lung collapse, also known as a pneumothorax. A pneumothorax can be life threatening. Another example is when large bore vascular access catheters such as dialysis catheters are placed. If a large bore catheter is not ideally secured, it can be accidentally pulled out in its entirety, placing a patient at risk for life-threatening hemorrhage.

Another disadvantage of using an adhesive-based securement device is when a particularly strong adhesive is used, the skin may be irritated or torn when the adhesive anchor is removed, a notable problem in elderly patients who are known to have thin sensitive skin. Also, if a catheter must be placed through diseased or otherwise irritated skin, the large adhesive footprint of adhesive-based securement devices can compromise skin healing due to lack of skin breathing. In these instances, a suture-based securement system is most ideal.

Yet another disadvantage of adhesive-based devices is their large base, requiring a large adhesive footprint on the skin to maximize securement to the skin. As a result, adhesive-based securement devices are not ideally suited for small spaces such as the neck, a common placement location for central venous catheters. Particularly problematic are patients with a short neck or a thick neck. Yet another disadvantage of adhesive-based securement devices is the fact that attachment to skin can be compromised in areas where significant motion takes place such as the crease of the elbow, also known as the antecubital fossa. In such regions, it is preferable to place a suture-based securement because adhesive-based systems tend to fail.

The traditional, time-tested, and overall stouter approach to catheter securement involves suturing a catheter to the skin by way of medical grade suture material placed through the skin using needles, with the suture loop-tied to the catheter. Suture securement is performed with a needle, typically curved, and a hand-held clamp-like device such as a needle driver. There are many advantage of this approach including a more secure anchoring of a catheter to the skin. With a more secure hold, the likelihood of catheters inadvertently being pulled out decreases. Suture securement of a catheter to the skin has other advantages as well. It requires a smaller skin footprint than adhesive-based securement systems, making it ideally suited for tight spaces such as the neck. The smaller footprint also makes it the ideal choice in instances where securement to underlying diseased skin cannot be avoided. Therefore, the smaller suture footprint allows the diseased skin to breathe, facilitating skin healing. Finally, in regions of the skin where excess movement occurs such as near joints, suture securement of a catheter proves a stouter hold to the skin as adhesive-based devices tend to fail quickly.

Despite all the advantages, suture securement of catheters to skin has two significant disadvantages which often preclude its use even in instances where it is more appropriate than adhesive-based systems. One, it is time consuming, involving multiple steps. Two, it places the practitioner and the patient at risk for accidental needle sticks, a particularly unsafe event. The invention described herein addresses these ailments to the currently available methods.

### BRIEF DESCRIPTION OF THE FIGURES

FIG. 1 is a top view of a standard triple lumen catheter hub where the three catheter lumens coalesce into one triple-lumen catheter tube.

FIG. 2 is a top view of one embodiment of the device.

FIG. 2B is an angled top open-book view of a clamshell device that is designed to close over and completely encase/cover a standard catheter hub of FIG. 1.

FIG. 2C is an angled top view of the clamshell closed over the catheter hub, locking the catheter hub within the device.

FIG. 3 is a top view of the device with the safety feature disabled, the deployment slider advanced, and the needles deployed under the skin.

FIG. 4 is a side view of the device with the needle deployed under the skin or other biological tissue.

FIG. 5 is a side view of the device with the needle deployed and the device angled such that the needle tip has been exposed. The needle is ready to be clamped near or at the tip by a surgical clamp and pulled/detached or otherwise disarticulated from the device.

FIG. 5B is an angled top view of the device with the needles deployed and driven through biological tissue. The device is also angled such that the needle tips have been exposed and are above the surface of the biological tissue. The distal most segments of the needles have been clamped by a curved surgical clamp.

FIG. 5C is an angled top view of the device in which the operator has disarticulated the needles from the device and pulled the needles through biological tissue, simultaneously exposing the attached suture and pulling the attached suture through biological tissue.

FIG. 6A is an angled top view of one surface of a device depicting two suture securement flaps, also herein referred to as hinged flaps, in an open position. Two sutures are also depicted coursing through biological tissue.

FIG. 6B is an angled top view of one surface of a device depicting two suture securement flaps in a closed or locked position, clamping and locking two sutures in place. The closed position of the suture securement flaps cause the formation of two closed-loop sutures, effectively securing the device to the biological tissue.

FIG. 6C is an angled top view of a device depicting two suture securement flaps in a closed position. Each securement flap is associated with a cutting feature such that excess suture material is cut away by the cutting feature of the device.

FIG. 7 is an angled top view of one surface of a device and an overlying catheter hub. The device contains two hinged loop flaps in an open position.

FIG. 7B is an angled top view of one surface of a device and an overlying catheter hub. The device contains two hinged loop flaps in a closed or locked position. The post of each hinged loop flap fits securely within the corresponding catheter hub loop. The closed or locked position of the hinged loop flaps secures the catheter hub to the device.

FIG. 7C is an angled top view of one surface of a device and a segment of catheter. The device contains a catheter securement flap, also herein referred to as a hinged catheter flap, in an open position.

FIG. 7D is an angled top view of one surface of a device in which the catheter securement flap is in a closed or locked position, effectively locking the catheter segment securely to the device.

### SUMMARY OF THE INVENTION

For a right handed practitioner, securing a vascular access catheter to the skin using the traditional approach involves the following steps:
1. Pick up needle driver with right hand
2. Pick up suture housing with left hand
3. Using the needle driver, grasp the curved suture and remove from suture housing, then discard housing
4. With left hand fingers, adjust the needle position on the needle driver
5. With right hand, drive the needle into the skin and let go of needle
6. Re-grasp the needle with the needle driver and pull the needle and suture all the way through the skin
7. Thread needle though the loop on the catheter hub
8. Place a spacer so that when the knot is tied, it is not tied too close to the skin
9. Tie knot
10. Grab scissors or scalpel
11. Cut the excess suture and needle off
12. Repeat steps 1-11 to place another suture through the second loop on the catheter hub

For a right handed practitioner, securing a drainage catheter to the skin using the traditional approach involves the following steps:
1. Pick up needle driver with right hand
2. Pick up suture housing with left hand
3. Using the needle driver, grasp the curved needle and remove from suture housing, then
discard housing
4. With left hand fingers, adjust the needle position on the needle driver
5. With right hand, drive the needle into the skin and let go of needle
6. Re-grasp the needle with the needle driver and pull the needle and suture all the way
through the skin
7. Place a spacer so that when the knot is tied, it is not tied too close to the skin
9. Tie knot
10. Grab scissors or scalpel
11. Cut the excess suture and needle off, leaving long enough tails to tie around catheter
12. Wrap suture tails around catheter several times
13. Tie knot
14. Grab scissors or scalpel
15. Cut excess suture

Note that most drainage catheters are secured to the skin using only a single suture. However, two sutures may be required for instances where a particularly secure hold is indicated. In these instances, a practitioner would have to repeat steps 1 through 15 above.

Using our device for suture-based securement of a catheter to biological tissue such as skin, described herein, fewer steps are required. Therefore, the device described herein lowers the procedure time for securing a catheter to biological tissues. Also, since direct needle handling with a practitioner's fingers is no longer part of the suturing process (steps 4 above), our device improves the overall safety profile of suture-based catheter securement.

The steps involved in deploying our suture-based securement device to secure any vascular or drainage catheter to the skin or other biological tissue include:
1. Picking up device with left hand
2. Removing safety cap (if any) with right hand, exposing the needle(s) that are on the device
3. Driving the needles through the skin with left hand, and angle device to expose the needle tips
above the surface of the skin
4. Picking up needle driver or other clamping device with right hand
5. Using the needle driver or other clamping device, grasp the curved needle(s) at or near their
tips and pull the sutures through the skin (both needles can be grabbed in the same step)
6. Snapping shut a device flap or fastener that permanently grasps the suture(s)
7. Grabbing scissors or scalpel
8. Cutting excess suture and needle(s)
9. Snapping shut a device flap or fastener that grasps the catheter hub or catheter segment

There is a clinical need for providing a secure catheter anchoring system using needles and sutures that can be applied safely and quickly, with fewer steps when compared to the traditional suture and needle approach. Accordingly, it is the objective of this invention to provide a device or series of devices that contain feature for allowing a practitioner the safe and rapid placement of a suture-based anchoring mechanism for catheter securement to biological tissue.

With suture securement, fewer patients will present to the emergency room or their doctors' office because their catheter was accidentally pulled out, and fewer catheter re-insertion procedures to correct this problem will be performed. With suture securement, fewer hospital inpatients will be subject to the risks of accidental catheter dislodgement. Therefore, with a stouter suture-based catheter securement system, fewer life threatening clinical situations will be encountered.

The details of additional embodiments are set forth in the accompanying drawings and the description below. Other features, objects, and advantages of the embodiments will be apparent from the drawings and detailed description, and from the claims.

### DETAILED DESCRIPTION OF THE INVENTION

Specific non-limiting embodiments are described herein to enable any person skilled in the art to make or use the invention. Various modifications to these embodiments will be readily apparent to those skilled in the art, and the generic principles described herein can be applied to other embodiments without departing from the spirit or scope of the invention. Thus, it is to be understood that the description and drawings presented herein represent a presently preferred embodiment of the invention and are therefore representative of the subject matter which is broadly contemplated by the present invention. It is further understood that the scope of the present invention fully encompasses other embodiments that may become obvious to those skilled in the art and that the scope of the present invention is accordingly not limited.

The devices provided herein are invasive insofar as one or more needles are used for securing a catheter or the devices to biological tissue. In various embodiments, the devices are used to minimize the time and steps required to safely secure a catheter to biological tissues, particularly the skin, using a needle and suture.

In some embodiments, the device is made from one or more synthetic or natural materials including but not limited to plastic, silicone, rubber, metal, and metal alloy.

In some embodiments, the device is rigid or semi-rigid and configured to allow a user to hold the device in one hand, right or left, such that the user can manually guide the device and needle placement.

In some embodiments, the device is rigid or semi-rigid and configured to serve as a needle driver, allowing the user to drive the needle or needles through biological tissues such as skin.

In some embodiments, the device is part of a catheter, and not removable or separable from the catheter, most typically occupying a location referred to as the catheter hub. However, the device may occupy a location other than the hub. The catheter can be a vascular catheter or any other type of catheter including drainage catheter, surgical catheter, etc., or any type of catheter that is placed though the skin or other biological tissues.

In some embodiments, the device is a standalone device that attaches to a catheter, most typically in the region of the hub. However, the device can be configured to attach to any part of a catheter, including separate from the hub.

In some embodiments, the device contains a catheter attachment zone or area, located within or on the device, which serves as the area of the device that secures, fits, attaches, or otherwise articulates the catheter to the device.

In some embodiments, the device is a standalone device that reversibly or irreversibly snaps, adheres to, clamshells, or otherwise can be attached to a catheter at the catheter hub or anywhere along the length of a catheter. The device operator can snap, adhere, clamshell, or otherwise attach the device to a catheter at the catheter hub or anywhere along the length of a catheter.

In some embodiments, there is a predetermined aperture, groove, cut-out, or other such feature that accommodates catheters of specific French sizes (diameters) such that when the device snaps or clamshells closed onto a catheter, or any hinged flap locks or closes onto a hub or catheter, the aperture, groove, cut-out, or other such accommodative feature prevents complete obliteration of the catheter lumen. The diameter, material composition, surface texture, and length of the aperture, groove, cut-out or other such accommodative device feature can be optimized in order to prevent catheter movement relative to the device.

In some embodiments, the device contains a suture securement or clutching area that reversibly or irreversibly holds suture material in order to form a closed-loop configuration that secures the device to biological tissues such as the skin after the suture has been pulled through biological tissues.

In some embodiments, the device size, shape, thickness, materials used and outer and inner surface characteristics are configured to minimize biological tissue damage and catheter damage, minimize catheter dysfunction such as kinking, maximize patient comfort, maximize operator comfort, and maximize suture and catheter hold.

In some embodiments, the device houses one or more needles and suture material within or on the device.

In some embodiments, the device serves as a needle holder and needle driver simultaneously. The device is configured such that the needle or needles are held in position by the device during the normal course of biological tissue needle penetration and repositioning, but allow for ease of needle disarticulation by the device user at the discretion of the device user.

In some embodiments, the device is configured to contain one or more needles that are exposed and protrude from the device in their resting state. In such embodiments, there are no sliders, levers, buttons, or other such methods which expose, extend, or advance one or more needles from the device. The device operator directs or drives these exposed needles through biological tissues, with the device itself serving as the needle holder, driver, and handle by which a user can selectively penetrate biological tissues. After through-and-through needle penetration of biological tissue has taken place, the device operator uses a separate instrument such as a surgical clamp to grasp the needle or needles, pulling them apart or away from the device, completely disarticulating the needle or needles from the device, and pulling the attached suture material through the biological tissue. By way of tying or fastening the suture, a closed-loop of suture is formed through biological tissue, and the device becomes secured to the biological tissue.

In some embodiments, the instrument used to grasp or clamp the needle or needles may be a specialized tool for this particular purpose, and may embody additional features that aid in performing or finalizing suture deployment, suture advancement through biological tissue, closed-loop suture formation, suture securement, and suture cutting or ligation. In some embodiments, the device is configured to contain one or more needles that are not exposed from the device in their resting state. The device operator deploys one or more needles by way of a slider, button, switch, lever, or other such mechanisms. With deployment, the needles are exposed and extend from the device. Upon partial or full extension of the needle or needles, the device operator can manipulate the device so as to puncture biological tissue with the needle or needles or otherwise drive the needle or needles.

In some embodiments, deploying one or more needles is achieved by way of a spring mechanism.

In some embodiments, deploying one or more needles is achieved by way of a manual or motorized push or sliding mechanism.

In some embodiments, one or more of the needles are held in place by an external or internal needle-gripping feature of the device while in the resting state. By way of a button, slider, switch, lever, or other such mechanisms on the device, the device operator can loosen the grip on one or more needles so as to allow subsequent disarticulation of a needle from the device. Loosening the grip on a needle can occur before or after needle penetration of biological tissues, but most typically after, and at the discretion of the device operator.

In one embodiment, a button, slider, switch, lever, or other such mechanism loosens the grip on all needles simultaneously.

In some embodiments, the needle or needles employed are straight. In other embodiments, the needle or needles employed are curved, angled, or otherwise hooked, allowing the device operator to both enter and exit biological tissues with a single smooth motion or a few motions. Needle curvature, size, thickness, orientation, shape, material may be optimized to facilitate operator comfort, ease of needle penetration of biological tissues, depth of needle penetration, and avoidance of damaging the catheter.

In some embodiments, multiple suture needles are used and are deployed simultaneously. In other embodiments, multiple suture needles are employed and are each deployed separately by the device operator. In such instances, the device operator may elect to leave one or more needles un-deployed indefinitely.

In some embodiments, the needle or needles can rotate about an axis, thus allowing the device operator to more selectively place the suture needle in a desired biological tissue location.

In other embodiments, the needle or needles cannot rotate about an axis, and are held by the device in a fixed position, angulation, or rotation.

In some embodiments, the needle or needles can be incrementally deployed and refracted by the device operator. In other embodiments, the needles can be incrementally deployed but not retracted by the device operator.

In some embodiments, the needle or needles have one or several deployed states which expose the needles to varying degrees relative to the device, at the discretion of the device user.

In some embodiments, the needles may be advanced individually, independent of one another, by the operator.

In some embodiments, the needle or needles are positioned, angled, rotated, sized, or otherwise configured such that they cannot inadvertently puncture and damage the catheter. Differing needle angulation, curvature, rotation, size, and/or location on the device are attributes that can be adjusted to achieve optimized device user comfort and safety, promote ease of device operation, and minimize risk of catheter damage.

In some embodiments, the catheter in the vicinity of the deploying needle or needles can be protected by a removable or permanent sleeve or guard affixed to the device that prevents the needle or needles from damaging the catheter.

In some embodiments, during needle deployment, the needle or needles are locked and unable to be retracted or otherwise pushed back into the device. In addition, the needle or needles may not be allowed to rotate along any axis.

In some embodiments, the needle or needles are deployable or positioned so as to allow the device operator to selectively penetrate biological tissue and facilitate exit of the needle or needles from biological tissue. Such device manipulation allows the device operator to grasp biological tissue with the device's needle or needles using a single or several motions. Additional motions may be required to grasp biological tissues with additional needles.

In some embodiments, the spacing between needles can be adjusted by the device user or predetermined, in order for the device to accommodate catheters of various diameters. Adjustable or predetermined spacing between needles can be used to optimize suture placement within biological tissues relative to the indwelling catheter. A suture placed too far from the catheter can cause unnecessary tension on the suture material and thus facilitate suture failure. A suture placed too close to a catheter risks catheter puncture by the needle used to place the suture. A suture placed too close to a catheter can also represent a weak tissue anchor due to its close proximity to the compromised tissue accommodating the catheter. A smaller inter-needle distance would be appropriate for deploying needles adjacent and around small bore catheters. A larger inter-needle distance would be appropriate for deploying needles adjacent and around larger bore catheters.

In some embodiments, the needle or needles may be angled relative to the axis of the device. Needle angulation is chosen to facilitate needle entry and exit from biological tissues while minimizing the number of steps and hand motions. Needles may be angled independent of one another or may be angled as a function of the angle of one or more adjacent or remote needles.

In some embodiments, the needle or needles are spaced, angled, or otherwise positioned to allow the device user to simultaneously advance all needles through biological tissues such as skin. In some embodiments, the needles are spaced, angled, or otherwise positioned to allow the device user to serially, one at a time, advance the needles through biological tissues such as skin. In some embodiments, the suture used in the device comprises of one or more materials, lengths, or thicknesses.

In some embodiments, the suture may be biocompatible and may comprise of a monofilament, a braided suture, a synthetic suture, of natural material, or configured of a combination of synthetic or natural fibers. The suture material may contain one or more features, forms, compositions, or characteristics useful in improving the overall profile of device securement to biological tissue. Such features may be optimized to increase friction, improve antimicrobial profile, increase tensile strength, or improve other attributes useful for maximizing device securement to biological tissues.

In some embodiments, the suture is attached to the device at one or more ends. The suture can then be tied, locked to the device via a locking mechanism, or otherwise fastened in position by the operator. By way of tying, locking, or fastening the suture, the suture becomes an uninterrupted loop anchor, securing the device to the biological tissue through which it courses.

In some embodiments, the suture is attached to more than one needle, looped through an uninterrupted ring or segment of the device, such that when the suture is tied, locked, or otherwise fastened to the device, the suture becomes a closed-loop anchor and the device becomes inseparable from the biological tissue through which the suture courses.

In some embodiments, suture may be tied to another suture forming a closed-loop anchor such that the device becomes fastened to biological tissues via an attachment to the device or via an uninterrupted ring or other such segment of the device.

In some embodiments, a device may employ two or more closed-loop suture anchor features to secure the device to biological tissue. In some embodiments, a device employs one closed-loop suture anchor feature to secure the device to biological tissue.

In some embodiments, the closed-loop suture can be cut by a scalpel or scissors so as to facilitate catheter removal from biological tissues when clinically appropriate and at the discretion of the device user. In some embodiments, the suture has a beaded or otherwise non-smooth texture or configuration so as to maximize hold, grip, or friction when the suture is locked or otherwise fastened to the device.

In some embodiments, the resting state of the device is such that one or more needles are not exposed. In such an unexposed or non-deployed device configuration, the needle or needles cannot be advanced through biological tissues. In such an un-deployed device configuration, the needle or needles are housed within the device as a safety feature in order to minimize or eliminate the risk of injury to the patient and device operator. In such an un-deployed device configuration, the needle or needles are housed within the device as a safety feature in order to minimize or eliminate the risk of needle contamination.

In some embodiments, the resting state of the device is such that only the needle tips are not exposed. In such an unexposed or non-deployed device configuration, the needle or needles cannot be advanced through biological tissues. In such an un-deployed device configuration, the tip of the needle or needles are housed within the device or flush with the device surface as a safety feature in order to minimize or eliminate the risk of injury to the patient and device operator.

In some embodiments, the device has one or more safety features such as a button, switch, slide, lever, or other such mechanism that must be pressed, switched, moved, slid, or otherwise engaged to allow exposure of one or more needles or needle tips. Such safety features minimize or eliminate the risk of injury to the device user and patient. Such safety features minimize or eliminate the risk of needle contamination.

In some embodiments, the device has one or more safety features such as a removable or non-removable cap, covering, lid, or other such needle barriers that must be removed, moved, slid, or otherwise engaged to allow exposure of one or more needles or needle tips. Such safety features minimize or eliminate the risk of injury to the device user and patient. Such safety features minimize or eliminate the risk of needle contamination.

In some embodiments, a device safety feature comprises of an embedded or attached needle covering or other barrier that must be slid, uncovered, rotated, or otherwise engaged in order to expose one or more needles or needle tips. The covering may be plastic, metal, glass, metal alloy, or other barrier material that minimizes or eliminates the risk of unintended puncture of biological material or needle contamination.

In some embodiments, the device does not have an embedded or otherwise fused barrier safety feature. Instead, one or more detachable or otherwise removable caps, lids, or other barrier coverings minimizes or eliminated the risk of unintended needle penetration of biological tissues or needle contamination.

In some embodiments, one or more of the detachable, removable, embedded, or otherwise fused barrier safety features may be plastic, metal, glass, metal alloy, or other material that can block unintended puncture of biological material or needle contamination.

In some embodiments, once trough-and-through needle and suture advancement has taken place through biological tissue such as skin, the device operator can secure the device to the biological tissue by tying together the suture material using traditional surgical knots.

In some embodiments, the device operator can fasten the suture material to a reversible or irreversible fastening or securement feature on the device, including by way of one or more hinged flaps. Such a suture securement forms a closed-loop suture. When the closed-loop suture passes through biological tissue such as skin, the closed-loop secures the device to biological tissue.

In some embodiments, the device operator can fasten or otherwise secure the suture material to a reversible or irreversible fastening or securement feature on the device, including by way of one or more hinged flaps, so as to allow for quicker and/or more secure fastening of the device to biological tissues.

In some embodiments, one or more suture securement flaps, also herein referred to as suture fastening flaps or hinged flaps, can secure, lock, fasten, or otherwise render immobile one or more sutures. Such a suture securement forms a closed-loop suture. When the closed-loop suture passes through biological tissue such as skin, the closed-loop secures the device to biological tissue.

In some embodiments, one or more suture securement flaps may reversibly or irreversibly secure, lock, fasten, or otherwise render immobile the suture material. In some embodiments, one suture securement flap may reversibly or irreversibly secure, lock, fasten, or otherwise render immobile all the suture material of a device.

In some embodiments, the suture securement flap or flaps may be located anywhere on the surface of or within the device. The suture securement flap or flaps may be any size, shape, configuration, or composition. In some embodiments, the suture securement flap or flaps also simultaneously secures locks, fastens, or otherwise renders immobile a catheter hub and/or segment of catheter.

In some embodiments, the suture securement feature of the device does not comprise of one or more hinged flaps. Instead, alternative methods of suture securement, locking, or fastening may be located on or within the device and employed by the device such as a grasping slit, post, cleat, or other means of suture securement, locking, or fastening.

In some embodiments, the suture securement feature of the device employs an adhesive or other means of increasing friction or grip between the device and suture material so as to form a more secure or otherwise stout closed-loop. The adhesive may be located on the suture or the device including device flaps. Other means of increasing frictional forces or grip between suture material and the device, including device flaps, may include changing the surface characteristics of the suture or the device, including device flaps.

In some embodiments, the suture fastening or securement feature of the device comprises of one or more hinged or winged fastener or securement flaps which close onto and hold one or more sutures, anchoring the sutures in place so as to prevent suture movement. Such hinged or winged suture fasteners or flaps may reversibly or irreversibly fasten one or more sutures.

In some embodiments, the suture fastening or securement feature comprises of one or more posts, cleats, or other structures around which suture is wrapped and consequently held in place to prevent suture movement. A complementary fastener, slit, or other holder designed to clinch or grip suture material may be incorporated so as to prevent suture unwinding from the one or more posts or cleats.

In some embodiments, the device contains a reversible or irreversible suture locking, fastening, gripping, or clinching feature, including by way of one or more hinged flaps, for allowing suture material securement to the device and secure closed-loop suture formation through biological tissue.

In some embodiments, the device contains a blade or other cutting apparatus to allow the operator to remove the needle or needles from the suture material. Such a blade or cutting apparatus may also be used for cutting excess suture material, leaving smaller or non-existent suture tails after device securement has been established. This blade or cutting apparatus may be sufficiently covered to prevent injury to the patient and device operator.

In some embodiments, one or more blades or other cutting apparatuses are located on the suture securement flaps or other hinged flaps such that suture is cut when the flaps close or lock. In some embodiments, the device operator can lock, fasten, or otherwise secure suture to the device, forming a closed suture loop, while simultaneously cutting the suture. Both securing suture and cutting suture can thus be performed in one motion or step.

In some embodiments, one or more blades or other cutting apparatuses are located on the device, including within or near the area of the suture securement flap or other flaps, such that when the securement flap closes over and secures suture, forming a closed suture loop, cutting of suture is simultaneously performed. Both securing suture and cutting suture can thus be performed in one motion or step.

In some embodiments, more than one blade or other cutting apparatuses are located on or within the device.

In some embodiments, more than one blade or other cutting apparatus is located on a suture securement flap, including a hinged flap.

In some embodiments, all the sutures coupled to a device can be cut in a single motion by one or more blades or other cutting apparatuses.

In some embodiments, the part(s) of the device that come in contact with a catheter segment is optimized such that movement of the catheter relative to the device is minimized and/or prevented. In these areas of the device, an adhesive may be used to limit catheter movement relative to the device. Alternatively, a rubber or other material that maximizes friction with the catheter may be used.

In some embodiments, fastening, securing, locking, or otherwise rendering immobile a segment of catheter relative to the device is accomplished in a device groove, cut-out, aperture, or other accommodative device feature. Such a groove, cut-out, aperture, or accommodative feature may be located anywhere on the device surface or within the device. The groove, cut-out, aperture, or accommodative feature may contain an adhesive or other means of increasing friction between the device and a catheter segment, meant to minimize movement or sliding of catheter relative to the device.

In some embodiments, a groove, cut-out, aperture, or other accommodative feature may contain additional materials meant to minimize catheter sliding or movement or increase frictional forces between the device and a catheter segment.

In some embodiments, a groove, cut-out, aperture, or other accommodative feature may be of such size, volume, length, or diameter so as to impart pressure on the outer surface of a catheter segment and to hold the catheter more securely, minimizing catheter sliding or movement. The pressure may be such that the shape or diameter of the catheter segment accommodates the shape and dimensions of the groove, cut-out, aperture, or other accommodative feature without obliterating the inner diameter of the catheter segment, although the inner diameter of the catheter segment may become smaller.

In some embodiments, the device surfaces that contact a catheter segment may be scored, undulated, beaded, roughened, or otherwise altered to increase frictional forces between the device and a catheter segment. Such surface characteristics may be employed in order to prevent sliding or movement of a catheter relative to the device.

In some embodiments, the device contains one or more zip ties designed to quickly wrap a catheter segment or catheter hub and prevent sliding or movement of the catheter or catheter hub relative to the device.

In some embodiments, a catheter securement flap, also herein referred to as a hinged catheter flap, is used to cover, lock over, snap over, close over, or otherwise secure one or more segments of catheter to the device in order to minimize catheter sliding or movement relative to the device.

In some embodiments, more than one catheter securement flap is present on a device.

In some embodiments, one or more catheter securement flaps may be located on any surface of or within the device.

In some embodiments, catheter securement flaps may be any size, shape, or composition.

In some embodiments, one or more catheter securement flaps may also secure, lock, close over, or otherwise render immobile suture material.

In some embodiments, one or more catheter securement flaps may also secure, lock, close over, or otherwise render immobile any part of a catheter hub including hub loops.

In some embodiments, one or more catheter securement flaps irreversibly locks, secures, closes, or otherwise snaps shut over a catheter segment.

In some embodiments, one or more catheter securement flaps reversibly locks, secures, closes, or otherwise snaps shut over a catheter segment.

In some embodiments, one or more catheter securement flaps contains a raised area that applies pressure to or increases frictional forces on a catheter segment when the flaps is closed or locked over a catheter. In other embodiments, more than one raised area on each catheter securement flap may be present. These raised areas may contain scored, undulated, roughened, beaded, or otherwise optimized surface characteristics to prevent catheter sliding or movement relative to the device. These raised areas may be of a material or contain material that increases frictional forces between the catheter and the device, including adhesive.

In some embodiments, one or more device flaps, including hinged flaps, hinged loop flaps, and hinged catheter flaps, reversibly closes, locks, shuts, or secures.

In some embodiments, one or more device flaps, including hinged flaps, hinged loop flaps, and hinged catheter flaps, irreversibly closes, locks, shuts or secures.

In the figures provided herein, [100] is the surface of biological tissue, most typically skin. [101] is a catheter that enters the surface of a biological tissue. In various embodiments, the catheter [101] is a single lumen, double lumen, or triple lumen catheter. [102] is a loop on the hub of a catheter that serves as the attachment point of suture material, securing the catheter hub to biological tissue, according to some embodiments and [103] is the catheter hub. In various embodiments, there are one or two hub loops [102] on a catheter or catheter hub. In various embodiments, a catheter may have no catheter hub [103] or hub loops [102].

In various embodiments, [104] is one of the ports of a triple lumen catheter, double lumen catheter, or single lumen catheter, [105] is the device and [106] is a safety slider. [107] is a needle deployment slider, in the un-deployed position and [108] is a needle deployment slider in the deployed position. In various embodiments, the needles have extended from the device.

In various embodiments, there is no safety slider or deployment slider, and the needle or needles extend from the device in their resting state, always ready to penetrate biological tissue. [109] is the needle still attached to device. [110] is suture material that has been exposed from within or on the device after the needle [111] has been disarticulated and pulled away from the device.

[112] is a suture securement flap, also herein referred to as a hinged flap. [113] is a suture cutting apparatus such as a blade or scissors. In various embodiments, the suture cutting apparatus may be located on any surface or location of the device, including one or more device flaps, hinged flaps, hinged loop flaps, or hinged catheter flaps.

[114] is a hinged loop flap for securing a catheter hub and/or hub loop to the device. In some embodiments, any single flap, including a hinged flap, can secure both suture material and catheter hub to the device.

[115] is a catheter securement flap, also herein referred to as a hinged catheter flap, which secures one or more segments of catheter to the device. In some embodiments, any single flap, including a hinged flap, can secure both suture material and one or more catheter segments to the device. [116] is the raised area on the catheter securement flap which applies some degree of pressure on a catheter segment when the catheter securement flap is in the closed or clamped position. In some embodiments, such pressure increases frictional forces between the catheter and the device, thus minimizing motion of the catheter relative to the device.

[117] is the groove, cut-out, or similar accommodative feature of the device allowing increased surface contact between device and catheter.

### DETAILED DESCRIPTION OF THE FIGURES

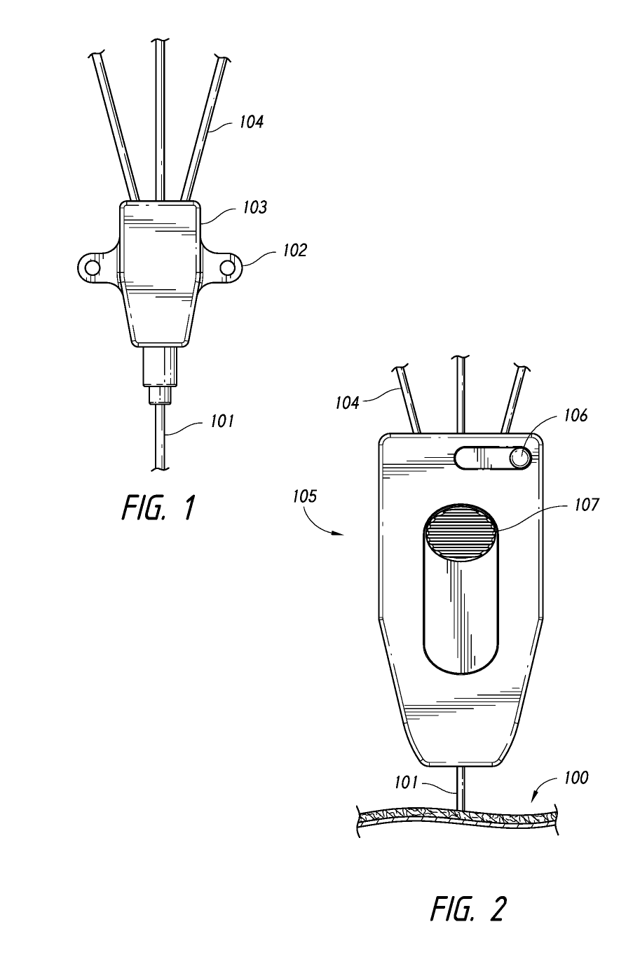

At least some embodiments of the medical devices disclosed herein can be handheld suture-based devices used to secure a catheter to biological tissue such as skin near/at the catheter exit site in order to minimize, inhibit, or prevent catheter movement relative to the biological tissue. The devices can decrease the time required to secure a catheter to biological tissues using suture. The devices can serve as a viable alternative to adhesive-based securement devices which are typically applied speedily and are therefore preferred to traditional suture securement. The devices can serve as a more reliable securement method than adhesive-based devices. Although the devices are discussed in connection with securing a catheter to biological tissues, the devices and methods disclosed herein can be used in other applications to achieve different results. Exemplary devices are discussed in connection with FIGS. 1-7D. FIG. 1 is a top view of a standard triple lumen catheter hub in which three catheter lumens [104] coalesce at the hub [103] into a single triple lumen catheter [101]. The single triple lumen catheter [101] is typically inserted into biological tissues such as skin. Standard double lumen catheters have two catheter lumens [104] which coalesce at the hub [103]. A single lumen catheter may also have a hub serving as a point of attachment to the catheter. The catheter hub [103] typically contains hub loops [102] which serve as anchoring points for suture-based or adhesive-based securement of the catheter hub [103] to biological tissues such as skin.

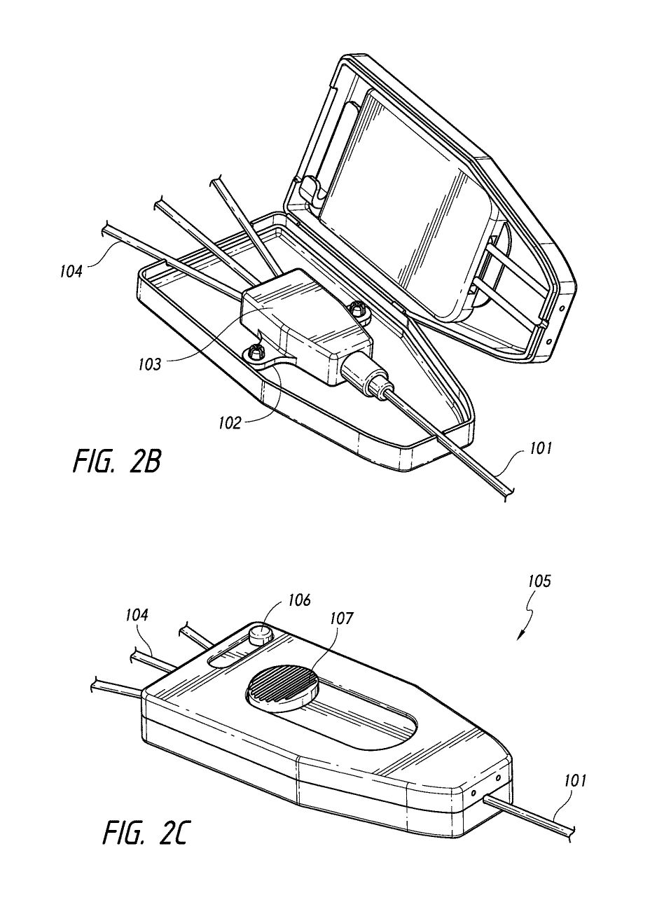

FIG. 2 is a top view of one embodiment of the device [105]. The device [105] is seen covering, enveloping, and attaching to a triple lumen catheter hub. In some embodiments, the device [105] does not completely cover or envelop a catheter hub, but only partially covers the hub. In other embodiments, the device [105] does not cover the hub. Instead, the device [105] covers, envelops, and/or attaches to one or more hub loops [102]. In FIG. 2, the three catheter lumens [104] are seen coursing toward the device [105]. The single triple lumen catheter [101] is seen emanating from the device [105] to enter biological tissue [100] such as skin. The device [105] can alternatively cover, envelope, or otherwise attach to a double lumen catheter or a single lumen catheter. The device [105] may comprise of any material including but not limited to plastic, rubber, other synthetic polymer or natural material, metal, metal allow, or a combination of materials including synthetic and natural materials.

In one embodiment, the device contains a safety feature such as a slider [106], button, switch, or other mechanism that must be pressed, slid, or otherwise engaged in order for an operator to deploy one or more needles. In FIG. 2, the pictured slider [106] has not been advanced, pushed, or otherwise engaged and therefore does not allow advancement of the needle deployment slider [107]. The pictured slider [106] is in the safe position. In some embodiments, the device does not contain a safety feature.

In one embodiment, the needle deployment mechanism consists of a deployment slider [107]. In FIG. 2, the deployment slider [107] has not been advanced, pushed, or otherwise engaged, and therefore the needle or needles have not been deployed. Alternatively, the needle deployment mechanism may consist of a button, lever, switch, or other mechanism that deploys one or more needles beyond the device when advanced, pushed, or otherwise engaged. Such deployment may involve one or more needles extending beyond the confines of the device or other conformational changes that exposes one or more needle tips for their subsequent advancement through biological tissues. In some embodiments, the device does not contain a deployment slider [107]. Instead, the device contains one or more exposed needles, ready for subsequent placement or driving through biological tissue.

FIG. 2B shows one embodiment of the device in which it opens to accommodate the catheter hub [103] positioned within the device. In this embodiment, the device opens in an open-book configuration. The hinges allow for opening to occur, and may be located anywhere on the device. In addition, the hinges may allow opening to any angle. Any component of the device [105] including device flaps or valves of a bivalve configuration may be of any size, shape, material composition, or configuration.

Any device surfaces that come in contact with the catheter hub [103] may be configured to securely grip the catheter hub [103]. This may consist of any material or surface configuration which increases friction and/or adheres to the catheter hub [103] including glue, tape, rubber, or other adhering material. In addition, posts, hooks, pegs, or other catchers may be positioned within or on the device to fit one or more hub loops [102] in order to grip the catheter hub [103] to the device. Any device surfaces that come in contact with a catheter segment [101] or [104] may be configured to securely grip the catheter. This may consist of any material or surface configuration which increases friction and/or adheres to catheter including glue, tape, rubber, or other adhering material.

FIG. 2C shows the closed-book view of the embodiment depicted in FIG. 2B. In this embodiment, the catheter hub is completely enclosed within the closed-book device [105]. The coalescing catheters [104] are seen in addition to the single triple lumen catheter [101]. In some embodiments, an internal latch or other locking mechanism secures the device in the closed-book. The latch or other locking mechanism may provide a reversible or irreversible locking feature. In some embodiments, an external latch or other locking mechanism secures the device in the closed-book configuration, providing a reversible or irreversible locking feature. A safety slider [106] is depicted in this embodiment and positioned in the safe position. The deployment slider [107] is in the undeployed position. In some embodiments, the device [105] may not contain a safety feature. In some embodiments, the device [105] does not have a deployment slider [107].

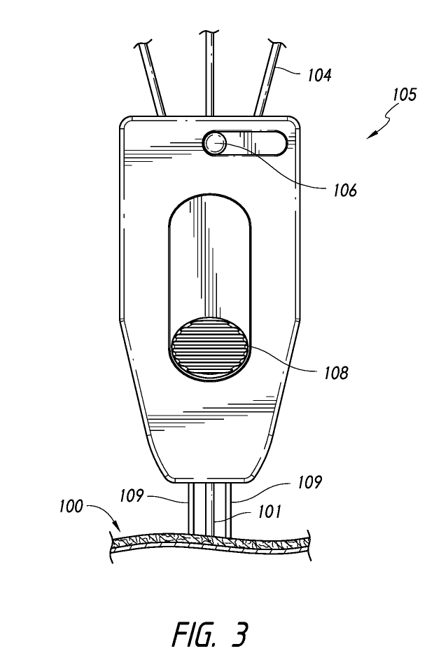

FIG. 3 is a top view of one embodiment of the device [105]. The device 105 is seen covering, enveloping, and attaching to a triple lumen catheter hub. The three catheter lumens

[104] are seen coalescing toward the device [105]. The single triple lumen catheter [101] is seen emanating from the device [105] to enter biological tissue [100] such as skin. In FIG. 3, the safety slider [106] has been advanced so as to allow the deployment slider [108] to be advanced. The deployment slider [108] has been advanced and is depicted in the deployed position. In FIG. 3, advancement of the deployment slider has resulted in advancement of two needles [109] which have been embedded into biological tissue [100] and remain attached to the device [105]. In some embodiments, only one needle is advanced or otherwise exposed with advancement of the deployment slider.

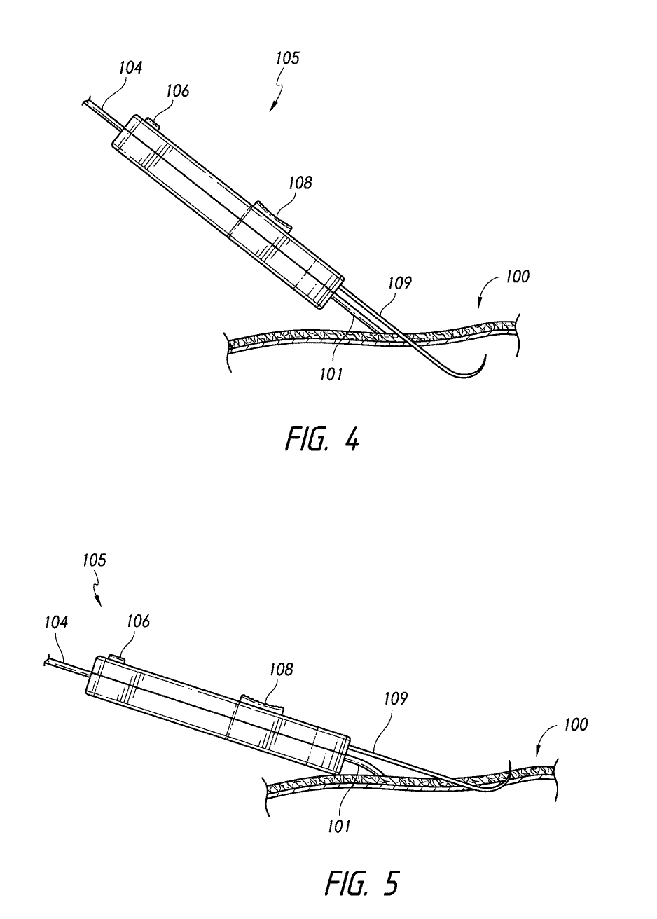

FIG. 4 is a side view of FIG. 3 in which the device [105] is positioned at an angle to biological tissue [100] such as skin. As depicted in FIG. 3, the safety slider [106] has been advanced, allowing the advancement of the deployment slider [108]. In FIG. 4, the deployment slider [108] is in the advanced position which has exposed a needle [109]. The needle [109] tip is seen traversing the surface of biological tissue [100]. The needle [109] is under the surface of the biological tissue. The catheter [104] enters the device [105]. The catheter [101] is seen exiting the device [105] and entering biological tissue [100]. The catheter [101] may be positioned anywhere relative to the needle [109] or needles of device [105], but is most typically positioned posterior to the needle [109] or needles. In some embodiments, device [105] contains two needles [109], and the catheter [101] may be positioned anywhere relative to the needles [109], but most typically between the two needles [109]. In some embodiments, the distance between catheter [101] and the needle [109] or needles is most typically between 2 and 5 millimeters, but can be any distance.

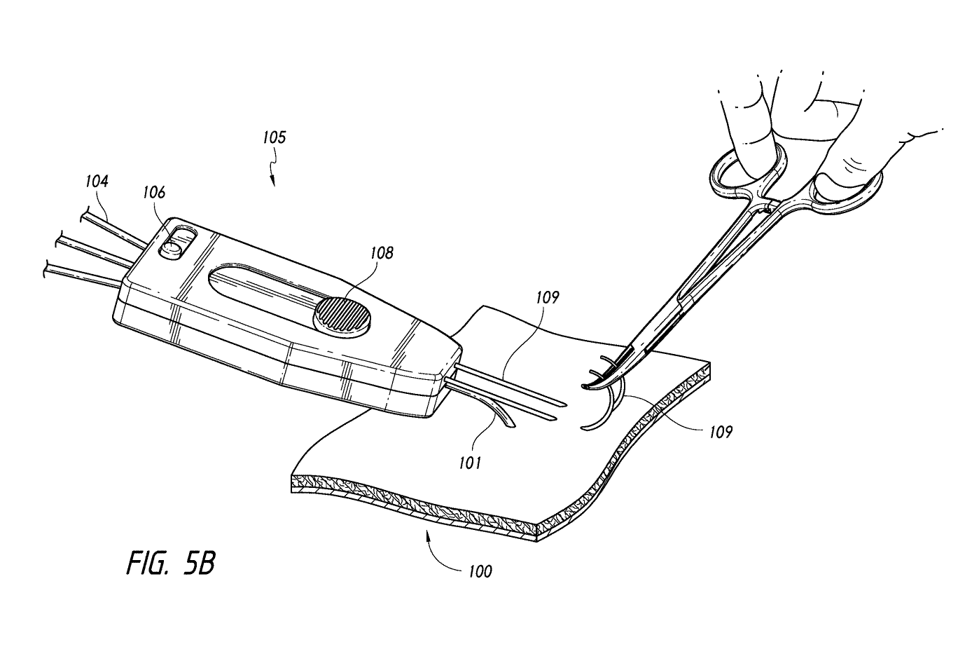

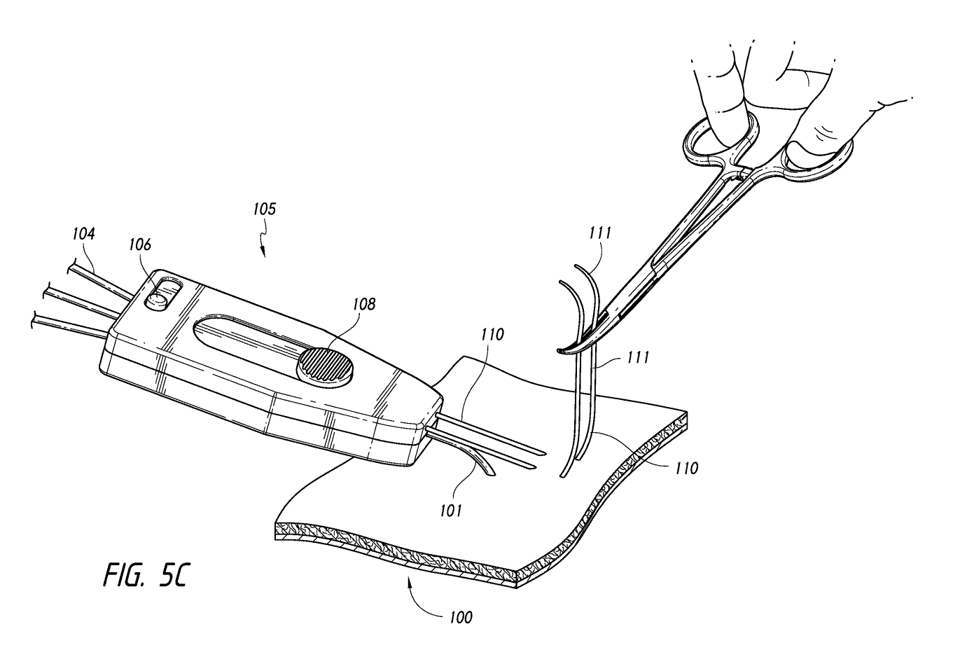

FIG. 5 is a side view of one embodiment of the device [105] in which the device [105] has been advanced closer to biological tissue [100] when compared to FIG. 4. In some embodiments, advancing the device [105] advances the needles [109] through the surface of biological tissue [100]. In some embodiments, advancing the device [105] advances the needles [109] deeper into biological tissue [100]. FIG. 5 also shows the device [105] at a smaller angle relative to FIG. 4, assuming a flatter overall position relative to biological tissue [100]. In some embodiments, the device [105] is positioned at a smaller angle to the surface of biological tissue [100] in order to expose the tip of one or more needles [109] from under the surface of biological tissue [100]. In some embodiments, the device [105] is rotated, twisted, pulled, or otherwise shifted in position in order to expose the tip of one or more needles [109] from under the surface of biological tissue [100]. In some embodiments, a detachable handle may be attached to the device [105] and used to manipulate the device [105] in order to advance or drive a needle tip or tips through biological tissue [100], or otherwise manipulate the device [105] to perform a device function.

FIG. 5B shows one embodiment of the device [105] in which the needles [109] have been directed through biological tissue [100]. The needle tips are exposed after having been advanced through biological tissue [100]. Near the needle tips, a curved surgical clamp is depicted grabbing or clinching the needles [109]. In some embodiments, a customized device is used to grab or clinch the needle [109] or needles. In some embodiments, the grabbing or clinching device also hides the tip of each needle such that accidental needle injury to the device operator is prevented. Also depicted is the catheter [101] which enters the biological tissue [100]. The catheter [101] may enter or exit biological tissue [100] at any position relative to the needles [109].

FIG. 5C depicts two needles [111] shown side by side which have been pulled through biological tissue [100] in their entirety, the needles [111] no longer being within the biological tissue [100]. Both needles are attached to or otherwise in continuum with the suture material [110] which is seen coursing through biological tissue [100]. The suture material [110] facilitates an ongoing physical attachment between the needles [111] and the device [105]. As the device operator pulls the needles through biological tissue [100], the suture material [110] becomes exposed from within the device [105]. In some embodiments, only certain lengths of suture material [110] are exposed, typically 10 centimeters. However, the device [105] may contain and ultimately expose suture material of any length.

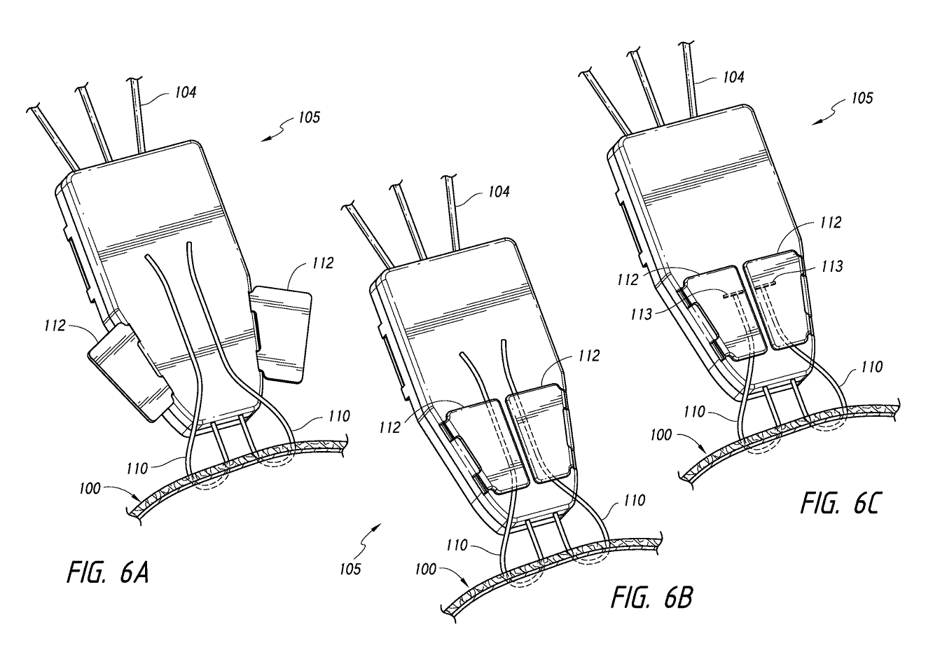

FIG. 6A depicts one embodiment of the device [105] with two hinged flaps [112] in an open position. The hinged flap or flaps [112] of the device [105] may be any size, shape, or composition. Suture material [110] is seen extending from the device and through biological tissue [100]. The suture material [110] is depicted looping through biological tissue [100] and back toward the device [105]. FIG. 6B depicts one embodiment of the device [105] with the two hinged flaps [112] in a closed and locked position. The hinged flaps [112] close and lock onto the suture material [110], preventing movement of the suture material and forming a closed-loop through biological tissue [100]. In some embodiments, the hinged flaps [112] contain additional or alternative securement features such as adhesive or a serrated surface which increases frictional forces between suture material [110] and components of the device [105] including the hinged flaps. Locking the suture material [110] to the device [105] may be accomplished by one or more hinged flaps [112] closing onto the suture material [110]. In some embodiments, other suture securement means such as a cleat, hook, or adhesive may additionally or alternatively be used. Each closed-loop secures the device [105] to biological tissue [100]. In some embodiments, the device [105] has one hinged flap that locks one or more sutures in place forming one or more closed-loops though biological tissue [100]. In some embodiments, more than two hinged flaps are located on the device [105] forming one or more closed-loops though biological tissue [100]. The hinged flap or flaps [112] may be located on any surface or location of the device [105]. Hinged flaps [112] may be located on multiple surfaces or locations of the device [105].

FIG. 6C depicts one embodiment of the device [105] in which the hinged flaps [112] are in a closed and locked position onto two sutures [110] forming two closed-loops of sutures [110]. The inner surface of the hinged flaps [112] contain a cutting feature [113] designed to cut suture material [110]. In some embodiments, the cutting feature [113] consists of a blade or scissor-like composite of two blades. The cutting features [113] may be located on any surface or location on the device [105], but is most typically on the inner surface of one or more hinged flaps [112]. In some embodiments, the motion of closing or locking one or more hinged flaps [112] allows for simultaneously cutting of suture material [110]. In other embodiments, a separate motion is required by the device operator to advance suture material across the cutting feature [113], advance the cutting feature [113] across suture material [110], or advance one or more blades across each other or the suture material [110].

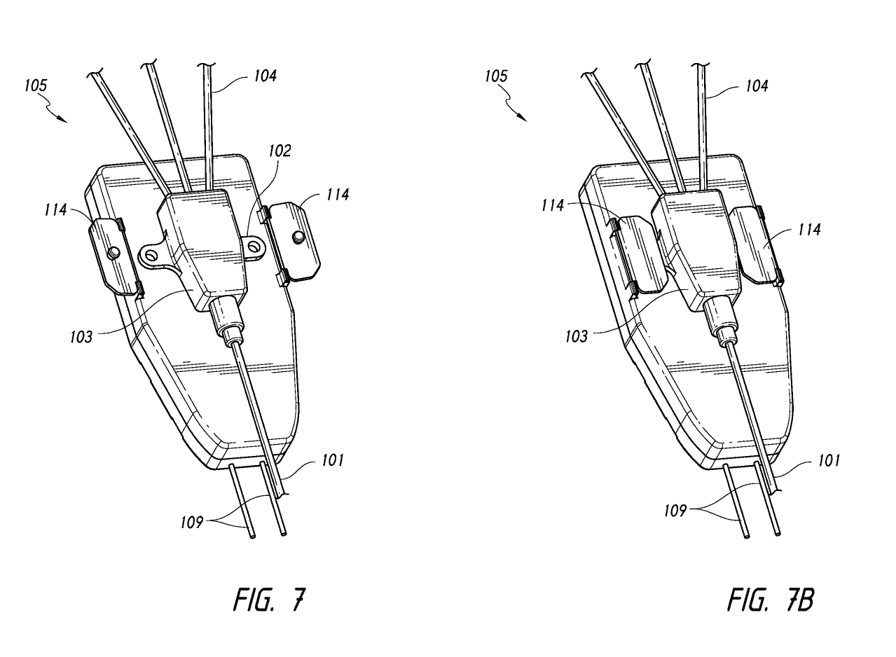

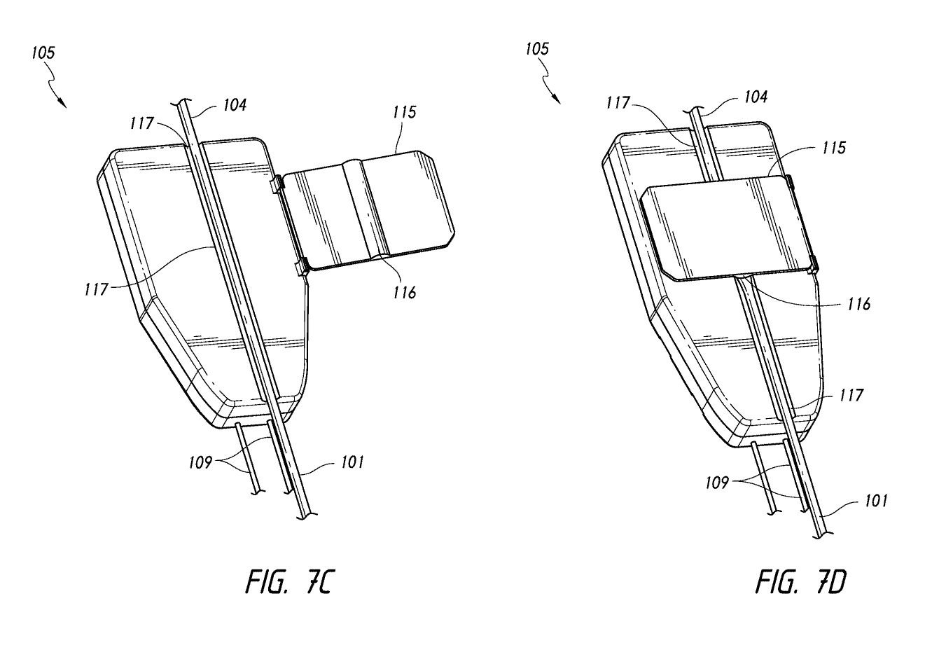

FIGS. 7 and 7B depict one embodiment of the device [105] in which the device securement to a catheter hub [103] occurs via one or more hinged loop flaps [114]. The hinged loop flaps [114] may be on any surface or location of the device [105], and may be any size, shape, or composition. In FIG. 7B, the hinged loop flaps [114] are depicted in a closed and locked position, securing the catheter hub [103] to the device [105]. In some embodiments, the hinged loop flaps [114] contain a post that fits through the hub loop [102] of the catheter hub [103] in order to more securely fasten the catheter hub [103] to the device [105]. In some embodiments, the hinged loop flaps [114] contain additional or alternative securement features such as adhesive or a serrated surface which increases frictional forces between the device [105] and the catheter hub loop [102] or catheter hub [103]. In one embodiment, a single hinged loop flap [114] secures the device [105] to the catheter hub [103] or hub loops [102]. In one embodiment, more than two hinged loop flaps [114] secure the device [105] to the catheter hub [103] or hub loops. In yet another embodiment, one or more hinged loop flaps [114] locks, fastens, or otherwise secures additional or alternative catheter components such as the catheter hub [103], and catheter segments [104] and [101]. In one embodiment of the device [105], one or more hinged flaps [112] or [114] assumes at least one physical feature or function of both the hinged loop flaps [114] and the hinged flaps [112]. In one embodiment of the device [105], one or more hinged flaps [112] assumes one or more of the physical features or functions described for the hinged loop flaps [114]. In one embodiment of the device [105], one or more hinged loop flaps [114] assumes one or more of the physical features or functions described for the hinged flaps [112].

FIGS. 7C and 7D depict one embodiment of the device [105] in which securement to a catheter [104] and/or [101] is depicted. The hinged catheter flap [115] is seen in an open configuration in FIG. 7C. The hinged catheter flap [115] is seen in a locked or closed position in FIG. 7D. The hinged catheter flap [115] may be any size, shape, or composition. The hinged catheter flap [115] may be located on any surface or location of the device [105]. In one embodiment, the device [105] contains a groove, cut-out, aperture, or other means to accommodate various lengths of catheter [104] or [101]. The groove, cut-out, aperture, or other accommodative area may be comprised of features designed to increase frictional forces on the catheter [104] or [101] in order to minimize movement of catheter [104] or [101] relative to the device [105]. Such features may include a serrated surface, adhesive, or other material such as silicone or rubber for the purpose of increasing frictional forces between the catheter [104] or [101] and the device [105]. In some embodiments of the device [105], one or more hinged catheter flaps [115] are present. One or more hinged catheter flaps [115] may contain raised areas [116] providing surface pressure on the catheter [104] or [101] when in the closed or locked position. The raised area [116] on the hinged catheter flap [115] may be any size, area, shape, or composition. The raised area [116] may contain materials that increase the friction with the abutting catheter [104] or [101]. In some embodiments of the device [105], the hinged catheter flap minimizes movement of the catheter [104] or [101] relative to the device [105]. In yet another embodiment of the device [105], no groove, cut-out, aperture, or accommodative means exist on the device [105]

In one embodiment of the device [105], one or more hinged flaps [112] or [115] assumes at least one physical feature or function of both the hinged catheter flaps [115] and the hinged flaps [112]. In one embodiment of the device [105], one or more hinged flaps [112] assumes one or more of the physical features or functions described for the hinged catheter flaps [115]. In one embodiment of the device [105], one or more hinged catheter flaps [115] assumes one or more of the physical features or functions described for the hinged flaps [112].

What is claimed is:

1. A suture-based catheter securement device, comprising:
a. one or more needles attached to or otherwise configured to be coupled to the device and that serve as the tissue-penetrating attribute of the device;
b. one or more needles attached to or otherwise configured to be coupled to the device such that a user can clinch the needles with an external clamp and pull the attached suture along with the needles through biological tissues such as skin;
c. suture material attached to the needle or needles serving as long-term loop anchors that course through biological tissues such as skin and hold the device in place relative to the biological tissues;
d. a rigid or semi-rigid configuration to allow a user to hold the device in one hand, right or left, such that the user can manually guide the device and needle placement into a subject's biological tissue;
e. a rigid or semi-rigid configuration to serve as a needle driver, allowing the user to drive the needle or needles through biological tissues such as skin;
f. one or more suture securement features, suture retainers, suture securement flaps, or hinged flaps configured to hold suture material such that the device can retain suture loops through biological tissues, holding the device in place relative to the looped biological tissues;
g. one or more catheter securement feature, catheter retainers, catheter securement flaps, hinged catheter flaps, or hinged loop flaps configured to hold and secure a catheter or catheter hub to the device, preventing any catheter movement relative to the device; and
h. one or more suture cutting features using one or more blades or other cutting apparatuses.

2. The suture-based catheter securement device of claim 1, wherein the needle or needles extend beyond the device in their resting position.

3. The suture-based catheter securement device of claim 1, wherein the needles are entirely located within the device and extend beyond the device at the user's discretion by way of a slider, button, lever, or other such manual, spring loaded, or automated means.

4. The suture-based catheter securement device of claim 1, wherein the needle or needles are spaced, angled, rotated, or otherwise positioned and coupled to the device in order to minimize risk of catheter puncture, maximize device operator comfort, and maximize use-efficiency whereby all the needles can be guided through biological tissues simultaneously or sequentially according to user preferences.

5. The suture-based catheter securement device of claim 1, wherein the needle or needles can be clinched by the device user with a separate clamping or clinching device and can be disarticulated and pulled away from the device, through biological tissues, thereby threading the attached suture through the needle path in the biological tissue.

6. The suture-based catheter securement device of claim 1, wherein one or all the needles can be clinched individually or simultaneously by the device user with a separate clamping or clinching device and can be disarticulated and pulled away from the device individually or simultaneously, thereby threading the attached suture through the needle path in the biological tissue.

7. The suture-based catheter securement device of claim 1, wherein the needle or needles are attached to suture material, all of which get pulled through biological tissues such as skin.

8. The suture-based catheter securement device of claim 1, wherein suture is attached to the device or to another needle through an uninterrupted segment of the device, thereby anchoring the device to biological tissue when closed suture loop or loops through biological tissue are formed.

9. The suture-based catheter securement device of claim 1, wherein the needle or needles, in part or in their entirety, are covered with a removable, embedded, or otherwise coupled cap, lid, covering, or other such barrier that minimizes or eliminates the risk of needle contamination or user or patient injury.

10. The suture-based catheter securement device of claim 1, wherein a safety button, switch, slide, lever, or other such mechanism must be pressed, switched, moved, slid, or otherwise engaged in order to allow advancement, exposure, or deployment of one or more needles or needle tips, thus minimizing or eliminating the risk of needle contamination or user or patient injury;

11. The suture-based catheter securement device of claim 1, wherein, after suture has been pulled through biological tissue, the suture can be locked, secured, or otherwise fastened to the device, forming one or more closed suture loops, thereby anchoring the device to biological tissue.

12. The suture-based catheter securement device of claim 1, wherein one or more securement features, suture retainers, suture securement flaps, or hinged flaps can be reversibly or irreversibly closed or locked in place thereby gripping suture material and forming one or more closed suture loops, thereby anchoring the device to biological tissue.

13. The suture-based catheter securement device of claim 1, wherein, after suture has been pulled through biological tissue, the suture can be locked, secured, or otherwise fastened to the device by way of tying sutures together, forming one or more closed-loops, thereby anchoring the device to biological tissue.

14. The suture-based catheter securement device of claim 1, wherein the coupled suture cutting feature comprises of one or more blades or cutting apparatuses located on the device.

15. The suture-based catheter securement device of claim 1, wherein the coupled suture cutting feature is located near, in close proximity to, or under the suture securement flap such that suture is cut simultaneously with suture securement.

16. The suture-based catheter securement device of claim 1, wherein the coupled suture cutting feature is located within or on the suture securement flap such that suture is cut simultaneously with suture securement.

17. The suture-based catheter securement device of claim 1, wherein the coupled suture cutting feature is located on the device in a position that allows simultaneous or sequential cutting of all coupled sutures.

18. The suture-based catheter securement device of claim 1, wherein any coupled suture cutting features are concealed, covered, or otherwise isolated such that the cutting surfaces do not allow the operator and the patient to be injured.

19. A suture-based catheter securement device, comprising:
a. a means for driving one or more needles through biological tissues in order to anchor the device to biological tissues, and wherein the means for pulling and disarticulating one or more needles away from the device for advancing the attached suture material through the needle-penetrated biological tissues;
b. a means for simultaneously driving multiple needles through biological tissues;
c. a means for sequentially, one at a time, driving needles through biological tissues;
d. a means for housing one or more needles within or on a device;
e. a means for housing one or more needles within or on a device that can be deployed or otherwise extended from the device, exposing the needle or needles in order to ready them for penetration of biological tissues;
f. a means for deploying or otherwise extending one or more needles from the device in order to ready them for penetration for biological tissues; and
g. a means for securing suture onto or within the device in order to form one or more loop anchors for securing the device to biological tissues.
h. a means for deploying, advancing, or otherwise exposing a needle or needles, simultaneously or one at a time;
i. a means for minimizing or eliminating the risk of needle contamination or user or patient injury by way of a removable, non-removable, or otherwise coupled needle barrier;
J. a means for minimizing or eliminating the risk of needle contamination or user or patient injury by way of requiring the device user to press, switch, move, slide, or otherwise engage a safety button, switch, slide, lever, or other such device couplings before needle deployment, exposure, or advancement can be initiated;
k. a means for cutting suture; and
l. a means for cutting suture and securing suture to the device simultaneously.

20. A suture-based catheter securement device of claim 19, wherein the means for driving one or more needles through biological tissues includes:
a. a rigid or semi-rigid device configuration that allows a user to hold the device in one
hand, right or left, and also allows the user to manually guide the device and needle placement into a subject's biological tissue; and
b. a rigid or semi-rigid device configuration to serve as a needle driver, allowing the user to drive the needle or needles through biological tissues such as skin.
